(19) United States
(12) Patent Application Publication
(10) Pub. No.: US 2007/0287976 A1
(43) Pub. Date: Dec. 13, 2007

(54) Adult incontinence management system

(76) Inventor: Ronald N. Sherrill, Knoxville, TN (US)

Correspondence Address: PITTS AND BRITTIAN P C, P O BOX 51295, KNOXVILLE, TN, 37950-1295, US

(21) Appl. No.: 11/451,700

(22) Filed: Jun. 13, 2006

(51) Int. Cl. A61F 13/15

(52) U.S. Cl. 604/385.06; 206/438

(57) ABSTRACT

A prepackaged kit containing various components each of which is useful in incontinent adult care. In a typical kit, the components are prepackaged into a single unit employing a disposable container and may include a pair of latex gloves, at least one cleaning wipe, a container of a protective ointment, and a fresh adult diaper. One or more of these components may be individually packaged. All of the components, including prepackaged components, are housed within the container which is clearly identified and which, in one embodiment, carries thereon a bar code useful in several administrative, financial or other functions.

### CROSS-REFERENCE TO RELATED APPLICATIONS

Not Applicable

### STATEMENT REGARDING FEDERALLY SPONSORED RESEARCH OR DEVELOPMENT

Not Applicable

### FIELD OF INVENTION

The present invention relates to the care of adult incontinence and particularly to a system which includes a kit containing disposable adult incontinence care components and packaging which may serve as a disposal receptacle of used adult incontinence care components.

### BACKGROUND OF INVENTION

In the field of infant and small child diapers, it is common to package multiple diaper-change components into a kit and offer the same for retail sale. These kits are purchased by parents of infants and/or small children, used only by the parent or caretaker of the infant or child, under conditions which offer little or no likelihood of contamination or cross-contamination of the parent, caretaker or the child. In general, homes are historically considered reasonably (or at least acceptably) asceptic. Further, there are only limited opportunities for non-residents of the home to expose an infant or small child to infection or other contamination in the course of changing of the diaper of the infant or small child. In any event, within the home, exposure is limited to a very select group of persons, including the parent, caretaker, and siblings. Likewise, there is even more limited likelihood that such non-resident will themselves be exposed to infection transmitted from the infant or small child in that such non-resident is not likely to be involved in the changing of the diaper of the infant or small child.

Further, there are no known laws nor regulations requiring that used infant or small child diapers used in the home by parents or caretakers be handled and disposed of as a contaminated material.

Contariwise, management of incontinence of adults residing in institutions of health or welfare care is governed by numerous laws and regulations. Even where laws and regulations relating to management of incontinence of adults are not applicable, good management practices dictate that the institution identify and take those actions to prevent, sources of irritation, pain, or contamination (including cross-contamination) associated with adult incontinence management. The handling and disposal of used adult diapers (also known as “briefs”) is paramount in adult continence management. (“diaper” and “brief” are used interchangeably in the present application) Institutionalized incontinent adults are particularly susceptible to irritation, infection, and similar risks known to be associated with improper incontinence management. Among the potential problems associated with the management of incontinence of institutionalized adults are selection of the proper type and size of brief, cleaning the patient's body, disposal of used product(s), and cross-contamination between the closely-quartered adults within a given institution, for example. The handling and disposal of used adult diapers is of specific concern. For example, such used diapers are to be handled and disposed of as a contaminated material. Disposal of such materials requires, among other things, that the used product be handled only by a trained and qualified attendant wearing latex gloves, that the used product placed in a clearly identified container which is suitably designed to receive contaminated materials preparatory to their disposal, and that the contents of these containers be disposed of by approved means, such as incineration. Understandably, the management of adult incontinence within institutions of health and welfare services are time-consuming and costly activities. Such costs normally must be accounted for in anticipation for reimbursement of the institution by insurance companies, governmental reimbursement, or other source of payment for such services.

Therefore, there is a need in the health and welfare care industry for a kit which provides the items needed for changing of the diaper for an incontinent adult and which further accommodates those ancillary matters such as costs, time of caretaker, and compliance with applicable laws, regulations and/or good management practices relating to handling and disposal of used adult incontinence products.

### BRIEF DESCRIPTION OF FIGURES

FIG. 1 is a representation of one embodiment of a kit embodying various of the aspects of the present invention, including a disposable bag useful for initial containment of fresh components for adult incontinence maintenance and subsequent disposal of soiled (contaminated) components;

FIG. 2 is a representation of a pair of latex gloves useful in one embodiment of the present kit;

FIG. 3 is a representation of an ointment package useful in one embodiment of the present kit;

FIG. 4 is a representation of cleaning wipes useful in one embodiment of the present kit;

FIG. 5 is a representation of an adult brief useful in one embodiment of the present kit;

FIG. 6 is a schematic exploded view of one embodiment of a kit of the present invention;

FIG. 7 is a schematic exploded view of a further embodiment of a kit of the present invention; and

FIG. 8 is a schematic exploded view of a still further embodiment of a kit of the present invention.

### SUMMARY OF THE INVENTION

In accordance with one aspect of the present invention, there is provided a prepackaged kit containing various components, each of which is useful in attending to the changing of an incontinent adult's diaper. In a typical kit, these components are prepackaged into a single unit and may include a pair of latex gloves, at least one cleaning wipe, a container of an protective ointment, and a fresh adult diaper. One or more of these components may be individually packaged. All of the components, including prepackaged components, are housed within a disposable container which, in one embodiment, among other things, carries thereon a bar code useful in several administrative functions, including patent usage data, financial accounting for the cost of the kit (and the accompanying diaper change procedure, as desired), among other information as desired.

### DETAILED DESCRIPTION OF INVENTION

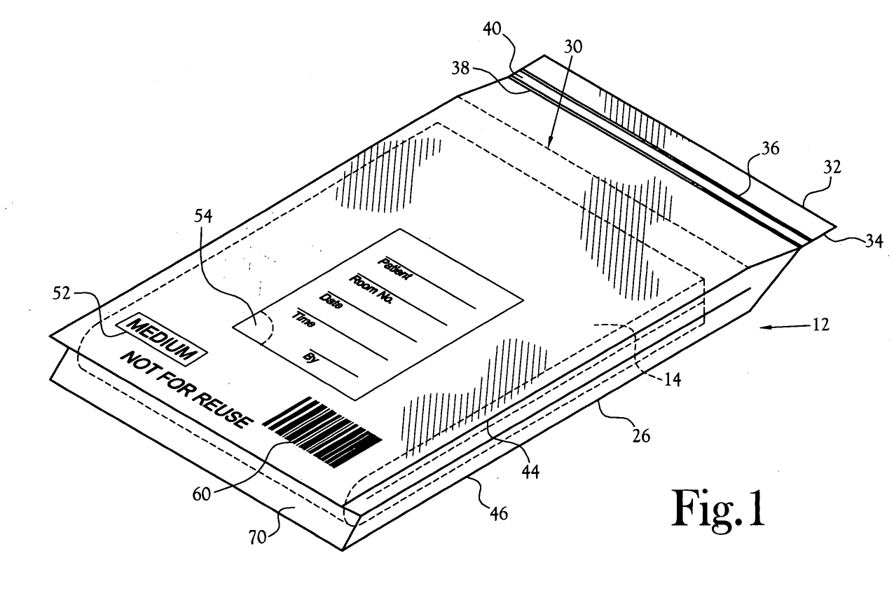

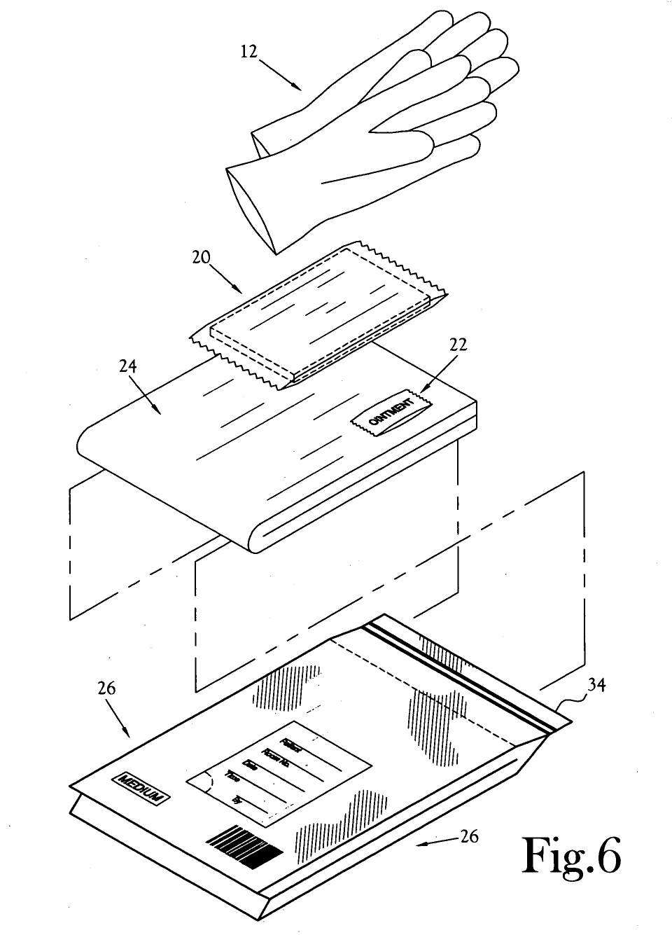

In accordance with one aspect of the present invention, there is provided a kit 12 containing preselected disposable components commonly known to be useful in the management of adult incontinence. One embodiment of a suitable kit is depicted in FIGS. 1-6 and includes at least (a) a pair of latex gloves 18, (b) at least one cleaning wipe 20, (c) a container of ointment 22, (d) a brief of a selected size 24, and, (e) a disposable bag 26 suitable for safe storage and handling of the kit contents and further for receiving and disposal of contaminated kit components.

In one embodiment, the disposable bag 26 serves to initially house the other components of the kit. This bag commonly is formed of a flexible polymeric film, at least partially, and preferably fully, transparent as permits visual observation of components housed in the kit. The bag further is designed to receive therein used (soiled and/or contaminated) components of the kit, and thereafter serve as a transport vehicle for transferring the bag and its contents to a proper disposal location. The bag captures therein both the used physical materials initially present in the bag and contaminants discharged by the patient. Moreover, the disposable bag may further inhibit release of odors associated with the soiled components.

Loading one or more of the components of the present kit into the bag is by way of the unsealed top end 34 of the bag. As depicted in FIG. 3, in one embodiment of the present invention, the fresh adult incontinence maintenance components of the kit are stacked atop one another in anticipation of their deposit within the bag. Preferably, the order of initial stacking of the components concurs with the reverse order of the common order of use of the components. That is, as depicted in FIG. 3, for example, the diaper 26 forms the bottom of the stack since this is the last component employed in an ordinary incontinence maintenance event. The next item in the stack is a container of ointment 22, which is the next to last of the components to be employed in an adult incontinence maintenance care event. Atop the container of ointment, there is stacked the container of wipes 20, followed in the stacking order by a pair of latex surgical-grade gloves 18.

Sealing of the open top end of the bag after loading it with its contents may be by means of one of several methods known in the art. In one embodiment, the bag is initially sealed across the width of the top end of the bag. At the top end of the bag, there is provided a tamper evident sealing tape which is initially covered by a pull tab. The bag further includes a tear line disposed inwardly of the top end of the bag. Opening of the bag to gain access to the contents is by means of the tear line. After any soiled materials are placed in the bag through the tear line opening, the pull tab is removed from the sealing tap, the top end of the bag is folded back upon itself to position the exposed sealing tape for engaging the side of the bag inwardly of the tear strip, and the sealing tape is employed to sealingly close the top end of the bag with the soiled contents inside the bag.

On the other hand, the bag may be made resealable through the use of interlocking ridges 36 and 38 and valleys 40 which serve to resealably close the top end of the bag. Such releasable sealing of the bag 26 is well known in the art and at times referred to as “zip lock” seals. In either of the noted embodiments for resealing the top end of the bag, the opposite side walls of the bag may be lightly heat sealed together at a location across the width of the bag outwardly beyond the contents of the bag and inwardly of the open end of the bag to define a tear line 30. This tear line serves to lightly, but securely bond the opposite side walls 44,46 of the bag to one another across the width of the bag. Rupture of the tear line does not adversely affect either of the side walls, but rather, such rupture merely effects separation of the side walls from one another. By this means, there is provided a tamper evident feature of the bag and the bag remains resealable employing either of the heretofore described means for sealing the top end of the bag. As noted, contaminated products may be placed into the bag through the open tear line, the open end of the bag may be resealed.

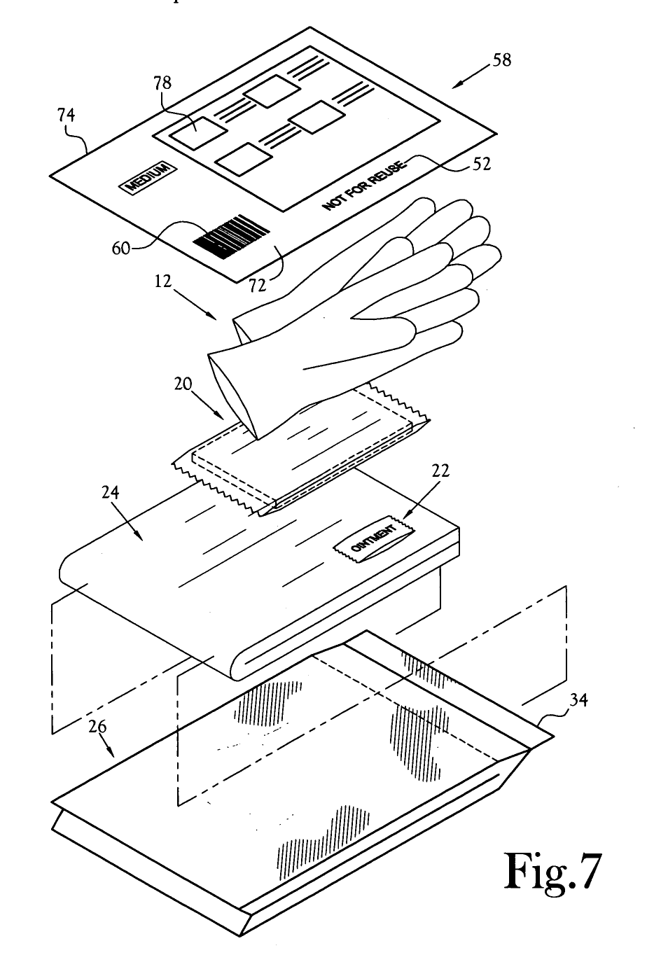

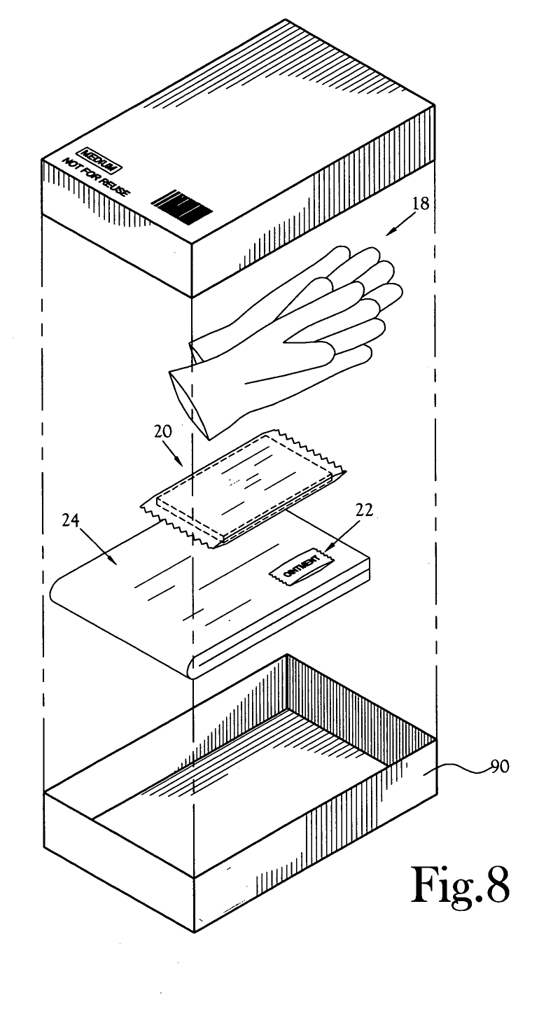

With reference to FIG. 7, depicting another embodiment of the present invention, a full sealing 50 of the bag across its open end 34 may be accomplished, for example, by heat sealing the opposite side walls of the bag to one another at the top end of the bag.

In the instance where the open end of the bag is fully sealed, the bag is not resealable after initially opening the bag to retrieve the contents therefrom. However, the opened bag remains available for the deposit of contaminated products therein for subsequent disposal.

Opening of the sealed bag to retrieve the contents thereof may be provided for by means of a line of perforations which extend through the thickness of each of the side walls of the bag as a location inwardly of the sealed end of the bag and which extend fully across the width of the bag. Rupture of the bag wall via these perforations fully opens the bag for retrieval of its contents.

In this embodiment, the bag need not be provided with a tamper evident feature inasmuch as access to the contents of the filled and sealed bag requires mechanical, and visually obvious, evidence of any tampering with the bag during storage and handling prior to its actual use in an incontinence management event.

The size of the disposable bag 26 is chosen to contain selected components of the present kit.

In the embodiment of the present invention depicted in FIG. 1, the bag is labeled. For example, the bag may be labeled 52 with the size, (small, medium, large, extra large, etc.) of the brief contained within the bag and/or a color code representative of the brief size may be applied to the bag. Such color coding is useful for ready identification of the kit from a distance, thereby aiding in ensuring that a given kit is to be used by a given patient. In one embodiment, the label includes a “peel off” portion 54 designed to permit a health provider to enter a patient's name, room number, time of day the kit was used, and the identification of the health care provider. As desired, removal of this “peel off” portion of the label may reveal underlying information (not shown) such as instructions as to proper means for use of the kit components and/or the proper means for disposal of the bag and used components of the kit contained within the bag.

Importantly, a bar code 60 may be applied to the bag 26. Among the data which can be included in the bar code are identification of the kit, including any one of many useful pieces of data such as size and component identifications, product number, manufacturer identification, and/or useful financial information.

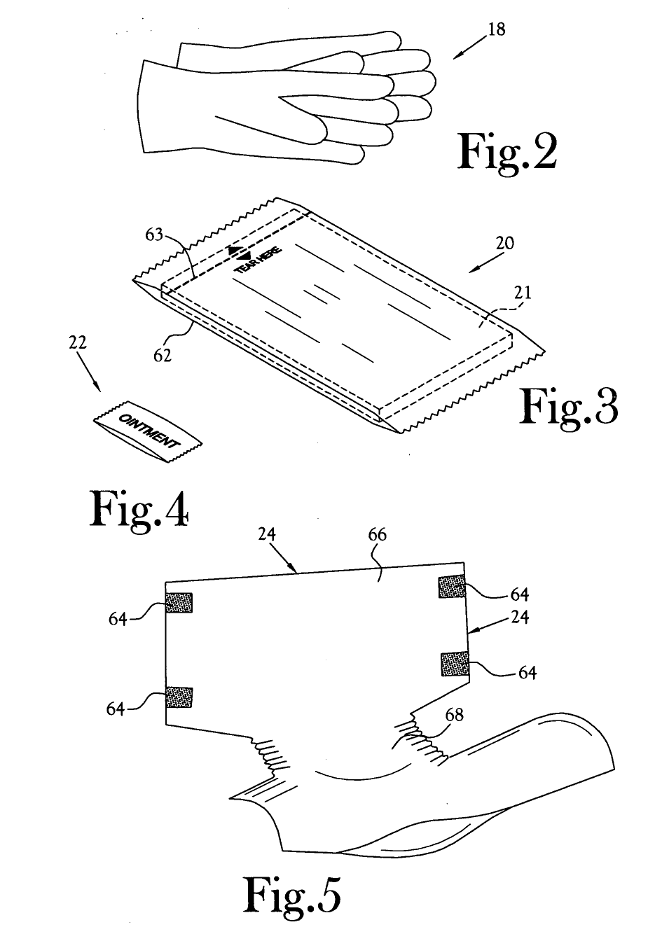

As depicted in FIG. 2, the depicted kit includes at least a pair of surgical-grade, powder-free latex gloves 18 such as those gloves which are commonly used in medical treatment environments.

The depicted kit further includes at least one, and preferably multiple, cleaning cloths (“wipes”) 21. Each cloth is of a size, such as 8″ by 10″, for example, sufficient to permit a health care provider to effectively remove any excrement which may remain on the patient's body after removal of a soiled brief. One suitable material for a wipe of the present invention is a spun-lace non-woven fabric. This material employs a single strands of thread entangled by high pressure water jets to give the effect of cotton made with staple fibers.

Preferably, each wipe is moisturized to assist in ready and complete clean-up of any portion of the patient's body which has become contaminated. In the kit of the present invention, the moisturized wipes preferably are packaged within a sealed moisture-proof package 62. This bag of wipes is included within the bag 26 of the present kit, thereby providing for maximum moisture retention of the wipes over extended storage periods. Their use is higly desirable over the use of common wash cloths using water and soap, the latter commonly creating an irritation to a patient. Moreover, the use of the present moisturized wipes reduces the time required for a health provider to effect a change from a soiled to a clean brief. In the depicted embodiment, the packaging for the wipes is provided with a peel-off tab 63 as an assist in opening the package of wipes.

Further, the kit includes a container, tube or sealed envelope, for example, of a measured quantity of a protective ointment suitable for a single-use change of the patient's brief. Employing this single-use quantity of ointment precludes any tendency for temporary storage and subsequent use of, a partially used tube of ointment which might have become contaminated during its initial use. Any of several protective ointments well known in the art may be employed in the present kit, such as petroleum jelly, antiseptic cream, or the like.

A single brief 24 is included in each kit. Through the use of a single brief per each kit, one can associate the number of kits used with the number of brief changes, hence the number of bladder or bowel evacuations experienced by a given patient over a given period of time, such as daily.

A suitable brief for use in the present kit comprises a common adult diaper (brief) such as are well known in the art. Each brief is of a soft flexible material or combination of materials designed to fit- an adult patient in the manner of a brief. The brief exhibits absorbency sufficient to retain at least a normal bladder discharge of a patient. In one embodiment, the brief may be provided with adjustable tabs 64 that provide for limited adjustment of the fit of the briefs, particularly in the waist 66 and leg 68 areas of the brief.

Typically, as noted, the components of a kit of the present invention are stacked in the order of their use. An alternative embodiment as depicted in FIG. 7, at least one side wall 46, for example, of the bag itself is substantially fully transparent and carries no or few labels. In this embodiment, this stack of components has overlaid on the top of the stack, a front sheet 58 of printable paperboard. This sheet is chosen to be of a size which is slightly less than the side 46 of the bag within which the stacked components are received. The sheet further is of a length which extends from proximate the closed bottom end 70 of the bag to a location proximate the top end 34 of the bag (or short of any tamper evident tear line or line of perforations as noted hereinabove) so that the sheet, as viewed through the transparent side wall of the bag takes on the appearance of a cover label for the bag, hence for the kit. This front sheet 58 includes first and second opposite surfaces 72 and 74, respectively, the first one 74 of which faces outwardly of the bag when the front sheet is disposed within the bag. This first surface of the front sheet is printed with various aspects of the kit, such as a listing 76, and identifying photograph 78, of each of the contents of the bag (with a brief description 80 of each component), a size label 52 for the diaper contained in the kit, a NOT FOR REUSE warning 82, a bar code 60, and/or like information useful to the health care user of the kit.

When disposed with the bag 26, the front sheet is disposed in overlying relationship of the stack of bag components, with its first surface facing the bag so that the information on the first surface of the front sheet is readily viewable through the transparent wall of the bag.

Further, preferably, the second (reverse) surface of the front sheet is printed with instructions (not shown) to the health care provider as to the manner of use of the various components of the kit.

In practice, when a health care provider determines that a patient is in need of a change from a soiled to a clean, new brief, the prepackaged bag 26 of the kit is opened, its contents are removed and laid out for ready access by the health care provider.

Thereupon, the health provider dons the latex gloves and proceeds to remove the soiled brief from the patient. Once the soiled brief is removed from the patient, it is folded from its outer perimeter inwardly upon itself and placed in the disposable bag 26. Employing the wipes, any contamination is removed from the patient's body and the used wipes are added to the bag 26. As needed, the tube of ointment is opened and a portion or all of its contents are applied to the patient's body as needed. The used tube and any remaining contents therein is thereupon added to the bag.

Finally, the health care provider removes the used latex gloves, adds them to the bag 26. Depending upon the choice of the closure for the top end of the bag, the health care provider may seal the top end of the bag employing the tape strip technique or by interlocking the ridges and valleys which extend across the width of the top end of the bag or other suitable closing means. This bag (whether sealed or not) and its contents are thereafter disposed of in accordance with accepted handling practices for contaminated material.

Through the use of the kit of the present invention, it is ensured that a given patient is provided with a properly sized brief which has not been tampered with subsequent to its inclusion in the kit. This assurance is enhanced by the use of a bar code applied directly to the disposable bag, to the first surface of the front sheet. Alternatively, the size of the brief contained within the kit may be coded through the use of color coding of the size label on the first surface of the front sheet and/or on the outer surface of the bag. Initial selection of a proper size brief for a given patient may include measuring of the patient and entry of a suitable size brief onto the patient's medical chart where such information is readily available to a health care provider for comparison with a kit which has been chosen for with by the given patient.

The bag and the components enclosed therein which comprise a kit of the present invention, is relatively flat and readily stored. As desired, in view of their relatively easy stacking property, an anticipated daily usage of the kits may be stored in a patient's room, thereby reducing the number of deliveries of kits from a central supply location to rooms of individual patient's. Being taper evident, the bags of the present kits discourage unauthorized use of a kit.

As noted, instructions for the use and disposal of each kit may be provided in the form of instructions printed on the second (inwardly facing surface of the front sheet) included in the kit, or by instructions printed on the outer surface of the bag of each kit.

Through the use of a kit of the present invention, savings are realized by reason of the prevention of “over use” of any one or more of the components in the kit. Further, presentation of known needed items in known useful quantities per each kit, underutilization of components of the kit, hence the possibility of less than adequate treatment for the patient, is minimized.

Whereas the present invention has been described using specific examples of components of a kit of the present invention, it will be recognized by one skilled in the art, that components other than and/or in addition to, the components specifically disclosed herein may be employed. Moreover, whereas packaging of the components of the kit in a bag is a preferred mode of presentment, it will be understood that a lidded disposable box 90 (see FIG. 7), properly designed as a disposal unit for contaminated material, may be employed in lieu of, or in addition to, the bag 26 disclosed herein. When the components of the kit of the present invention are contained in a box, without using a disposable bag, as desired, a front sheet 48 may be included within the box.

What is claimed:

1. A prepackaged kit useful in connection with adult incontinence maintenance comprising
at least a pair of latex gloves,
at least one cleaning wipe,
a container of a protective ointment, and
a fresh adult diaper,
a disposable container suitable for storage, handling and disposal of one or more contaminated medical products; and,
said components being contained initially within said disposable container.

2. The prepackaged kit of claim 1 wherein said disposable container comprises a bag formed of a polymeric film which is heat sealable.

3. The prepackaged kit of claim 2 and wherein said bag includes a resealable end, said resealable end including one or more sets of ridges and valleys extending across the width of said bag adjacent said resealable end and defining a resealable closure for said resealable end of said bag.

4. The prepackaged kit of claim 1 and including a tamper evident tear line formed across the width of said disposable container adjacent and inwardly of said resealable closure for said resealable end of said bag.

5. The prepackaged kit of claim 1 wherein one or more of said components is individually packaged prior to being added to the kit.

6. The prepackaged kit of claim 1 and including indicia displayed on an outer surface of said disposable bag and which identifies said kit as non-reusable.

7. The prepackaged kit of claim 1 and including a bar code carried on said disposable bag, said bar code being useful in several administrative functions, including usage, financial accounting for the cost of the kit and its use in an incontinent adult maintenance function.
